(19) United States
(12) Patent Application Publication
(10) Pub. No.: US 2026/0297011 A1
(43) Pub. Date: Oct. 1, 2026

(54) METHODS FOR PRODUCTION OF BUTYRIC ACID

(71) Applicant: SUPERBREWED FOOD, INC., New Castle, DE (US)

(72) Inventors: Brian WAIBEL, Kennett Square, PA (US); Bryan P. TRACY, Wilmington, DE (US); Aharon EYAL, Jerusalem (IL)

(21) Appl. No.: 19/480,813

(22) PCT Filed: May 7, 2024
(86) PCT No.: PCT/IB2024/054407

§ 371 Date: Nov. 2, 2025

Related U.S. Application Data
(60) Provisional application No. 63/464,580, filed on May 7, 2023.

(51) Int. Cl. C07C 51/34; C07C 51/43; C07C 51/44

(52) U.S. Cl. CPC C07C 51/34; C07C 51/43; C07C 51/44

(57) ABSTRACT

Provided is a method of producing butyric acid comprising providing a fermentation liquor comprising butyrate, acetate, at least one counter ion, and at least one fermentation co-product, at a butyrate to acetate weight/weight ratio of 2 and 20, pH between 5.0 and 8.0, and combined concentration of butyrate and acetate between 20.0 and 75.0 wt %; combining the fermentation liquor with a mineral acid having a concentration of greater than 30 wt %, wherein the mineral acid forms with the counter-ion a first mineral salt with water solubility at 25° C. greater than 25 wt %, whereby an acidulation product comprising an organic phase comprising butyric acid, acetic acid and water, and an aqueous phase comprising the first mineral salt is formed, wherein a pH of the aqueous phase is between 2.0 and 4.7; separating the two phases to form an organic phase and an aqueous phase; and separating butyric acid from the organic phase.

### CROSS-REFERENCE TO RELATED APPLICATION

The present application gains priority from U.S. Provisional Application No. 63/464,580 filed May 7, 2023 which is incorporated by reference as if fully set-forth herein.

### FIELD OF THE INVENTION

The present invention relates to methods of producing butyric acid, and more specifically to such methods comprising (i) providing a fermentation liquor comprising water, butyrate, acetate, at least one counter ion, and at least one fermentation co-product, wherein (a) a butyrate to acetate weight/weight ratio is in the range between 2 and 20, (b) a pH is in the range between 4.5 and 8.0, and (c) a combined concentration of butyrate and acetate is in a range between 20.0 and 75.0 wt %; (ii) providing a mineral acid solution comprising the mineral acid and water, wherein (a) the concentration of the mineral acid is at least 70 wt % and (b) the mineral acid is characterized by forming with the counter-ion a first mineral salt with water solubility at 25° C. greater than 25 wt %; (iii) combining the provided fermentation liquor with the provided mineral acid, whereby an acidulation product is formed, which acidulation product comprises at least two phases, an organic phase comprising butyric acid, acetic acid and water, and an aqueous phase comprising water and the first mineral salt of the acid and the counter ion, wherein a pH of the aqueous phase is in a range between 2.0 and 4.7; (iv) separating the two phases to form a separated organic phase and a separated aqueous phase; and (v) separating butyric acid from the separated organic phase to form separated butyric acid.

### BACKGROUND

Fermentation broth containing butyrate and acetate can be refined via various methods, such as nanofiltration, reverse osmosis, and base treatment. Base treatment could include treatment with ammonium hydroxide, sodium hydroxide, or calcium hydroxide (as milk of lime).

Nanofiltration is sensitive to the cutoff size of the membrane used, requiring the use of nanofiltration membranes with pore sizes in the range of 100-150 Dalton to enable concentration of butyrate and acetate in the retentate. Furthermore, butyric acid has a molecular weight in this cutoff range. The molecular weight of acetic acid is less than the 100 Dalton cutoff and, thus, nanofiltration results in significant loss of acetic acid to the permeate.

As the pH of the solution approaches the pKa of these organic acids, the loss of organic acids to the permeate is amplified. Furthermore, this method has limited ability to concentrate the retained butyrate and acetate, as the ability to concentrate the solution is limited by the pressure rating of the nanofiltration membrane, which is currently in the range of 600-1000 psi (40-69 bar).

Reverse osmosis (RO) provides somewhat better retention of butyric acid and acetic acid. This method is limited by the rated transmembrane pressure of the reverse osmosis membrane of the pump and piping system within which the membrane is applied. Current technology limits the pressure to 600-1000 psi (40-60 bar). The retentate concentration is restrained by the balance between the RO system's rated pressure and the osmotic pressure of the resultant solution. This limits the concentration of the butyric acid to approximately 10 g/l (~1 wt %).

Base treatment converts the butyrate and acetate to their salt forms (ammonium butyrate/ammonium acetate for ammonium hydroxide, sodium butyrate/ammonium acetate for sodium hydroxide, and calcium butyrate/ammonium acetate for calcium hydroxide). The salt is recovered via evaporation and recrystallization from water. Ammonium is a weak base, so the evaporation process generally involves loss of ammonium during evaporation. Sodium hydroxide is a strong base. The sodium butyrate/sodium acetate salt can be recovered, with effort from solution. This method involves substantial energy input and a solid that tends to accumulate on the surfaces of the process equipment. Calcium hydroxide treatment creates the calcium salt of the organic acids. This method is commercially used in citric acid and lactic acid manufacturing, as calcium citrate and calcium lactate have very limited solubility in water and, thus, the salt readily precipitates from aqueous solution and forms crystal structures that can be readily filtered. Calcium butyrate and calcium acetate have much higher solubility in water, exceeding that of sodium chloride in water. Thus, the calcium hydroxide treatment offers little benefit over sodium hydroxide.

Once the salt from base treatment is recovered, the salt must be contacted with mineral acid, typically sulfuric acid or hydrochloric acid. This recreate a concurrent salt (sodium sulfate or sodium chloride in the case of sodium hydroxide treatment or calcium sulfate or calcium chloride in the case of calcium hydroxide). Calcium sulfate is gypsum and has limited industrial use. These salts are waste streams that require disposal or careful handling to upgrade them for use in food ingredients.

There is thus an unmet need for an improved method for the production of butyric acid which is devoid of at least some of the disadvantages of the prior art methods.

### SUMMARY OF THE INVENTION

According to an aspect of some embodiments of the present invention, there is provided comprising (i) providing a fermentation liquor comprising water, butyrate, acetate, at least one counter ion, and at least one fermentation co-product, wherein (a) a butyrate to acetate weight/weight ratio is in the range between 2 and 20, (b) a pH is in the range between 4.5 and 8.0, and (c) a combined concentration of butyrate and acetate is in a range between 20.0 and 75.0 wt %; (ii) providing a mineral acid solution comprising the mineral acid and water, wherein (a) the concentration of the mineral acid is at least 70 wt % and (b) the mineral acid is characterized by forming with the counter-ion a first mineral salt with water solubility at 25° C. greater than 25 wt %; (iii) combining the provided fermentation liquor with the provided mineral acid, whereby an acidulation product is formed, which acidulation product comprises at least two phases, an organic phase comprising butyric acid, acetic acid and water, and an aqueous phase comprising water and the first mineral salt of the acid and the counter ion, wherein a pH of the aqueous phase is in a range between 2.0 and 4.7; (iv) separating the two phases to form a separated organic phase and a separated aqueous phase; and (v) separating butyric acid from the separated organic phase to form separated butyric acid.

### DETAILED DESCRIPTION OF THE INVENTION

The present invention relates to methods of producing butyric acid comprising (i) providing a fermentation liquor comprising water, butyrate, acetate, at least one counter ion, and at least one fermentation co-product, wherein (a) a butyrate to acetate weight/weight ratio is in the range between 2 and 20, (b) a pH is in the range between 4.5 and 8.0, and (c) a combined concentration of butyrate and acetate is in a range between 20.0 and 75.0 wt %; (ii) providing a mineral acid solution comprising the mineral acid and water, wherein (a) the concentration of the mineral acid is at least 70 wt % and (b) the mineral acid is characterized by forming with the counter-ion a first mineral salt with water solubility at 25° C. greater than 25 wt %; (iii) combining the provided fermentation liquor with the provided mineral acid, whereby an acidulation product is formed, which acidulation product comprises at least two phases, an organic phase comprising butyric acid, acetic acid and water, and an aqueous phase comprising the first mineral salt of the acid and the counter ion, wherein a pH of the aqueous phase is in a range between 2.0 and 4.7; (iv) separating the two phases to form a separated organic phase and a separated aqueous phase; and (v) separating butyric acid from the separated organic phase to form separated butyric acid.

The combination of fermentation liquor concentrated to greater than 30 wt % and a mineral acid results in the creation of a mineral salt with very limited solubility in the organic acid. This forces a phase split that concentrates the organic acids in a lower density layer and the salt and most of the water in a higher density aqueous layer. Further, in the presence of fermentation liquor concentration of 35, 37, or 40 wt % or higher, the resultant salt exceeds the solubility limit in water and results in precipitated solid. This precipitate forms large, rapidly settling agglomerate solids that are highly permeable. The slurry readily settles from the balance of the aqueous layer and the organic layer. The interstitial fluid readily drains from the agglomerated solids. This slurry layer can be efficiently dried, as there is low water retention with the precipitated salt. Further, since the aqueous layer is saturated with salt, a recrystallization process can be readily used to remove the water from this layer. Finally, the extremely low solubility of the salt in the organic acid results in significant reduction in water in the organic layer to less than 30, 26, 25, 24, 23, 22, 15, 12 wt %, or lower water content in the organic layer. The acids are more than 96% and as much as 98% retained in the organic layer, resulting in minimal loss of butyric and acetic acid products to the aqueous and slurry layers.

The present inventors have surprisingly found significant differences in the effect of temperature on retention of butyric acid and acetic acid in the organic layer, which would not be expected by one of ordinary skill in the art.

Butyric acid retention in the organic layer is greater than 90% at 25° C. if the water content of the feed solution is less than 55 wt. % water. This retention of butyric acid in the organic layer is relatively unaffected by temperature. For example, at 60° C., the level of butyric acid retention in the organic layer is approximately the same as that at 25° C. On the other hand, the solubility of acetic acid is more strongly temperature-dependent. At 25° C. and at a feed water content of 55 wt. % or less, more than 50% of the acetic acid is retained in the organic layer. At 60° C. and water content of 55 wt. % or greater, the acetic acid retention in the organic layer increases to around 55%. Both the insensitivity to temperature on the butyric acid retention in the organic layer and the temperature sensitivity of the acetic acid would be considered counter-intuitive to one of ordinary skill in the art.

Furthermore, the combined effect of water content and temperature on distribution of acetic acid between the aqueous and organic layers would be considered counter-intuitive. The present inventors have surprisingly found that at lower water content levels (less than 50 wt % in the feedstock), increases in temperature result in an increased acetic acid distribution in the organic layer, while at higher water content, temperature increase results in a reduction of acetic acid distribution in the organic layer.

The particulars shown herein are by way of example and for purposes of illustrative discussion of the various embodiments of the present invention only and are presented in the cause of providing what is believed to be the most useful and readily understood description of the principles and conceptual aspects of the invention. In this regard, no attempt is made to show details of the invention in more detail than is necessary for a fundamental understanding of the invention, the description making apparent to those skilled in the art how the several forms of the invention may be embodied in practice.

The present invention will now be described by reference to more detailed embodiments. This invention may, however, be embodied in different forms and should not be construed as limited to the embodiments set forth herein. Rather, these embodiments are provided so that this disclosure will be thorough and complete, and will fully convey the scope of the invention to those skilled in the art.

Unless otherwise defined, all technical and scientific terms used herein have the same meaning as commonly understood by one of ordinary skill in the art to which this invention belongs. The terminology used in the description of the invention herein is for describing particular embodiments only and is not intended to be limiting of the invention.

As used herein, the term “fermentation co-product” refers to proteins, peptides, amino acid, vitamins, and salts that are either minor metabolites required for fermentation, extrudates of the cultivated microorganism, or constituents of the microorganism resulting from cell lysis during handling separation of cellular biomass from fermentation liquor.

As used herein, the term “acidulation product” refers to the reaction products resulting from the addition of mineral acid to a fermentation liquor.

As used herein, the term “refining” refers to removal of at least 85 wt % impurities.

As used herein, the singular forms “a,” “an,” and “the” are intended to include the plural forms as well, unless the context clearly indicates otherwise.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements. Every numerical range given throughout this specification will include every narrower numerical range that falls within such broader numerical range, as if such narrower numerical ranges were all expressly written herein. Additional advantages of the invention will be set forth in part in the description which follows, and in part will be obvious from the description, or may be learned by practice of the invention. It is to be understood that both the foregoing general description and the following detailed description are exemplary and explanatory only and are not restrictive of the invention, as claimed. As used herein, when a numerical value is preceded by the term “about”, the term “about” is intended to indicate +/−10% of that value.

As used herein, the terms “comprising”, “including”, “having” and grammatical variants thereof are to be taken as specifying the stated features, integers, steps or components but do not preclude the addition of one or more additional features, integers, steps, components or groups thereof. These terms encompass the terms “consisting of” and “consisting essentially of”.

According to an aspect of some embodiments of the present invention, there is provided a method for producing butyric acid, comprising
(i) providing a fermentation liquor comprising water, butyrate, acetate, at least one counter ion, and at least one fermentation co-product, wherein (a) a butyrate to acetate weight/weight ratio in the fermentation liquor is in the range of between about 2 and about 20, (b) a pH of the fermentation liquor is in the range of between about 4.5 and about 8.0, and (c) a combined concentration of butyrate and acetate in the fermentation liquor is in a range of between 20.0 and 75.0 wt. %;
(ii) providing a mineral acid solution comprising the mineral acid and water, wherein (a) the concentration of the mineral acid is at least 70 wt % and (b) the mineral acid is characterized by forming with the counter-ion a first mineral salt with water solubility at 25° C. greater than 25 wt %;
(iii) combining the provided fermentation liquor with the provided mineral acid, whereby an acidulation product is formed, which acidulation product comprises at least two phases, an organic phase comprising butyric acid, acetic acid and water, and an aqueous phase comprising the first mineral salt, wherein a pH of the aqueous phase is in a range of between about 2.0 and 4.7;
(iv) separating the two phases to form a separated organic phase and a separated aqueous phase; and
(v) separating butyric acid from the separated organic phase to form separated butyric acid.

According to some embodiments, the counter ion is selected from the group consisting of an ammonium ion, a sodium ion, a potassium ion, a calcium ion and combinations thereof.

According to some embodiments, the fermentation co-product is selected from the group consisting of flagellar protein, other protein, peptides, and amino acid comprised of methionine, cystine, lysine, leucine, isoleucine, threonine, valine, histidine, glycine, aspartic acid, serine, glutamic acid, alanine, and tyrosine.

According to some embodiments, the butyrate to acetate weight/weight ratio in the fermentation liquor is about 2, about 3, about 4, about 5, about 6, about 7, about 8, about 9, about 10, about 11, about 12, about 13, about 14, about 15, about 16, about 17, about 18, about 19 or about 20.

According to some embodiments, wherein the method uses batch fermentation, the butyrate to acetate weight/weight ratio in the fermentation liquor is in the range of from about 2.2 to about 2.9, more preferably in the range of from about 2.4 to about 2.6.

According to some embodiments, a pH of the fermentation liquor is about 4.5, about 5.0, about 5.5, about 6.0, about 6.5, about 7.0, about 7.5 or about 8.0.

According to some embodiments, a combined concentration of butyrate and acetate in the fermentation liquor is about 20, about 25, about 30, about 35, about 40, about 45, about 50, about 55, about 60, about 65, about 70 or about 75 wt %.

According to some embodiments, the mineral acid is selected from the group consisting of sulfuric acid, phosphoric acid, nitric acid, hydrochloric acid and combinations thereof.

According to some embodiments, a concentration of mineral acid in the fermentation liquor is at least about 70 wt %, such as about 70 wt %, about 75 wt %, about 80 wt %, about 85 wt %, about 90 wt %, about 95 wt %, or greater than about 95 wt %. According to some embodiments, the concentration in the fermentation liquor is about 70 wt % for nitric acid; 85 wt % for phosphoric acid; and 98 wt % for sulfuric acid. The mineral acid should preferably be near its solubility limit in water.

According to some embodiments, the mineral acid forms with the counter-ion a first mineral salt with water solubility at 25° C. greater than about 25 wt %, such as about 30 wt %, about 35 wt %, about 40 wt %, about 45 wt %, about 50 wt %, about 55 wt %, about 60 wt %, about 65 wt %, or about 70 wt %.

According to some embodiments, a pH of the aqueous phase of the acidulation product is about 2.0, about 2.2, about 2.4, about 2.6, about 2.8, about 3.0, about 3.2, about 3.4, about 3.6, about 3.8, about 4.0, about 4.2, about 4.6 or about 4.7.

According to some embodiments, the combined amount of water in the provided fermentation liquor and the provided mineral acid is such that the concentration of the first mineral salt in the acidulation product is at least 80% of saturation at 25° C.

According to some embodiments, a water content in the provided fermentation liquor is in the range of from about 40% to about 50%, such as about 40%, about 41%, about 42%, about 43%, about 44%, about 45%, about 46%, about 47%, about 48%, about 49% or about 50%.

According to some embodiments, providing the fermentation liquor comprises fermenting a carbon source with cells of a microorganism capable of producing butyrate and acetate at a pH in a range between 4.5 and 7.5 and separating the cells of the microorganism, such as by centrifugation and/or microfiltration to form the fermentation liquor. According to some embodiments, butyrate is produced at a titer of between 2 and 60 g/L and acetate is produced at a titer of between 1 and 30 g/L at a pH in a range between about 4.5 and 7.5 (such as about 4.5, about 5.0, about 5.2, about 5.4, about 5.6, about 5.8, about 6.0, about 6.2, about 6.4, about 6.6, about 6.8, about 7.0, about 7.2, about 7.4 or about 7.5).

According to some embodiments, the carbon source is selected from the group consisting of a carbohydrate, such as a starch, cellulose, xylan or a sugar (such as glucose, xylose, galactose and combinations thereof); a sugar alcohol (such as 1,5-anhydroglucitol, erythritol, galactinol, lyxitol, myo-inositol, pentitol, sorbitol, threitol, xylitol and combinations thereof); a sugar acid (such as galactonic acid, glyceric acid, lactobionic acid, ribonic acid, saccharic acid, threonic acid, xylonic acid and combinations thereof) or a metabolite (such as glutrate, Acetyl-Coa, lysine and combinations thereof).

According to some embodiments, the microorganism is selected from the group consisting of a bacterium of the class Clostridia, such as of the order Clostridiales or Eubacteriales, optionally of the genus Ruminococcus, Clostridium, Eubacterium, Coprococcus, Subdoligranulum, Butyricicoccus, Roseburia, Pseudoflavonifractor, Flavonifractor, Oscillibacter, Faecalibacterium, Pseudoflavonifractor, Oscillibacter, Flavonifractor, Holdemanella, or Sarcina, and preferably of the species Eubacterium hallii, Eubacterium rectale, Clostridium butyricum, Faecalibacterium prausnitzii or Clostridium tyrobutyricum.

According to some embodiments, the method further comprises removing water from the fermentation liquor, such as by reverse osmosis or single or multi-effect evaporation. According to some embodiments, the fermentation liquor is concentrated to a total solute content in excess of 30 wt. %.

According to some embodiments, the acidulation product further comprises crystals of the first mineral salt, wherein the aqueous phase is saturated with the first mineral salt.

According to some embodiments, the acidulation product further comprises at least one selected from the group consisting of butyramide, amino acids of methionine, cystine, lysine, leucine, isoleucine, threonine, valine, histidine, glycine, aspartic acid, serine, glutamic acid, alanine, and tyrosine. According to some embodiments, the aqueous phase is saturated with the first mineral salt and some or no crystals of the first mineral salt.

According to some embodiments, the method further comprises adding a second mineral salt to the fermentation liquor such that the mixture of the combined fermentation liquor and second mineral salt has a water content of less than 60 wt %. According to some embodiments, the second mineral salt is the same salt as the first mineral salt. According to some embodiments, the second mineral salt is a different salt from the first mineral salt.

According to some embodiments, the method further comprises refining the separated organic phase to remove at least one impurity, such as by ultrafiltration, nanofiltration, or low temperature distillation, prior to separating butyric acid. According to some such embodiments, refining comprises removing proteins and peptides from the organism, removal of water via zeolite absorbance or low temperature distillation, or combinations thereof. According to some embodiments, separating butyric acid comprises distillation, extraction or a combination thereof.

According to some embodiments, the method further comprises removing water from the separated butyric acid, such as by adsorption, for example using a molecular sieve.

According to some embodiments, the method further comprises separating acetic acid, from the separated organic phase to form separated acetic acid, such as by distillation, extraction or a combination thereof.

According to some embodiments, the method further comprises separating the first mineral salt from the separated aqueous phase.

According to some embodiments, the method further comprises separating the crystals from the acidulation product to form separated crystals of the first mineral salt.

According to some embodiments, the counter ion is ammonium, wherein the mineral acid is sulfuric acid and the first mineral salt is ammonium sulfate.

According to some embodiments, the method further comprises reacting the separated butyric acid or a product thereof with hydrogen to form butanol. The separated butyric acid disclosed herein has been found to be sufficiently pure to enable such a reaction to occur, being substantially free of impurities that would otherwise deactivate hydrogenation catalysts.

According to an aspect of some embodiments of the present invention, there is provided a product comprising butyric acid produced as disclosed herein. According to some embodiments, the butyric acid in the product has a concentration of greater than about 50 wt %. According to some such embodiments, the butyric acid has a purity of greater than about 97%, such as about 98%, 99% or even about 100%. According to some such embodiments, a weight/weight ratio of butyric acid/acetic acid in the product is at least twice that in the provided fermentation liquor, such as twice, three times, four times, five times, six times, seven times, eight times, nine times, ten times or even higher than ten times that in the provided fermentation liquor.

According to a further aspect of some embodiments of the present invention, there is provided a product comprising the separated butyric acid as disclosed herein.

According to a further aspect of some embodiments of the present invention, there is provided a product comprising the separated acetic acid as disclosed herein.

According to a further aspect of some embodiments of the present invention, there is provided a product comprising the separated crystals of the first mineral salt as disclosed herein.

### EXAMPLES

### Example 1

Fermentation liquor comprising water, butyrate, acetate and lactate with a counter-ion of ammonium was concentrated by thermal evaporation under vacuum to concentrations of 349 g/L, 122 g/L, and 10.3 g/L of butyrate, acetate, and lactate, respectively. The solution further contained 6.44 wt. % of organic solids other than butyrate, comprising amino acids and salts from fermentation. The initial pH of the concentrate was 6.37.

Three batches having different pH values were prepared from this feedstock as follows:

### Batch I:

25.01 g of feed was combined with 4.33 g of concentrated sulfuric acid (93 wt. % purity). The addition was carried out slowly under active agitation and cooling to ensure that the temperature of the batch did not exceed 60° C. This resulted in a pH of 3.57.

After stopping agitation, the mixture separated in three phases: a light organic phase, rich in butyrate; an aqueous phase rich in ammonium sulfate and water and relatively deplete in butyrate; and a slurry phase.

The slurry phase comprised ammonium sulfate with the aqueous continuous phase. The masses of the phases were 13.61 g (48%) for the organic phase and, 14.76 g (52.0%) for the combined aqueous liquid and aqueous slurry. Furthermore, the aqueous liquid phase constituted 12.27 g (43.3%) of the resultant mixture while the aqueous solids (aqueous slurry on a dry basis) constituted 2.49 g (8.8%).

The butyrate preferentially shifted to the organic phase. The organic phase comprised 96.5% of the butyrate, 76.3% of the acetate and 61.2% of the lactate. The aqueous liquid phase comprised the balance of the materials (3.5%, 23.7% and 38.8% for butyrate, acetate and lactate, respectively).

### Batch II:

25.02 g of feed was combined with 4.81 g of 93 wt. % sulfuric acid. Acid addition was carried out under active agitation and cooling, as for batch I. This resulted in a pH of 2.99. After stopping agitation, the solution phase split as for batch I. The masses of the phases were 12.62 g (43.1%) for the organic phase, 16.64 g (56.9%) for the combined aqueous liquid and aqueous slurry. Furthermore, the aqueous liquid phase constituted 14.43 g (49.3%) of the resultant mixture while the aqueous solids (dry basis) were 2.21 g (7.5%).

As with batch I, the butyrate preferentially shifted to the organic phase. The organic phase comprised 95.8%, 70.9% and 54.5% of the butyrate, acetate and lactate, respectively. The aqueous liquid phase comprised the balance of the materials-4.2%, 29.1% and 45.6% for butyrate, acetate and lactate, respectively.

### Batch III:

31.01 g of feed was combined with 5.99 g of 93 wt. % sulfuric acid. Acid addition was performed under both active agitation and cooling as batch I. This resulted in pH 2.62. After stopping agitation, the solution phase split as in batch I. Mass split between the phases was 16.67 g (43.1%) for the organic phase, 16.64 g (56.9%) for the combined aqueous liquid and aqueous slurry. Furthermore, the aqueous liquid phase constituted 14.43 g (49.3%) of the resultant mixture while the aqueous solids (dry basis) constituted 2.21 g (7.5%).

The organic phase comprised 97.5%, 78.3%, and 62.8% of the butyrate, acetate and lactate, respectively. The aqueous liquid phase comprised the balance of the materials: 2.5%, 21.7% and 37.2% for butyrate, acetate and lactate, respectively.

Tables 1 and 2 below summarize the mass split and the component split, respectively of the three batches:

TABLE 1
Mass split

| Test condition | High pH | Medium pH | Low pH |
|---|---|---|---|
| Test pH | 3.57 | 2.99 | 2.62 |
| Organic phase | 48.0% | 43.1% | 46.6% |
| Aqueous phase | 52.0% | 56.9% | 53.4% |
| (liquid + solid) | | | |
| Liquid | 43.3% | 49.3% | 3.8% |
| Solid (dry basis) | 8.8% | 7.5% | 16.6% |

TABLE 2
Mass split

| Test condition | High pH | Medium pH | Low pH |
|---|---|---|---|
| Test pH | 3.57 | 2.99 | 2.62 |
| Organic phase | | | |
| Butyric acid | 96.5% | 95.8% | 97.5% |
| Acetic acid | 76.3% | 70.9% | 78.3% |
| Lactic acid | 61.2% | 54.4% | 62.8% |
| Aqueous phase | | | |
| Butyric acid | 3.5% | 4.2% | 2.5% |
| Acetic acid | 23.7% | 29.1% | 21.7% |
| Lactic acid | 38.8% | 45.6% | 37.2% |

In all three batches, aqueous liquid and aqueous slurry phases rapidly separated from the organic phase. The organic phase from each batch was subsequently processed via distillation to produce a purified butyric acid.

### Example 2

The effect of water content of the feedstock on the retention of butyric acid and acetic acid in the organic layer was studied. The feedstock containing 2.38:1 butyric acid to acetic acid with a 45 wt. % water content was subjected to serial dilutions with water and acidulated to a pH of less than 3.5 with 93 wt. % concentrated sulfuric acid.

The composition of the undiluted feed is shown in Table 3. Solutions were maintained at 15° C.

TABLE 3
Composition

| | |
|---|---|
| Ammonium butyrate | 36.3% |
| Butyric acid | 0.29% |
| Ammonium acetate | 16.4% |
| Acetic acid | 0.10% |
| Ammonium lactate | 1.4% |
| Lactic acid | 0.0% |
| Water | 45.4% |

Table 4 shows the mass split between the organic phase and the aqueous phase and further identifies the presence of any solid precipitate in the aqueous phase.

TABLE 4
Mass split

| Water content in | | | | | | |
|---|---|---|---|---|---|---|
| feed | 45.4% | 51.8% | 55.6% | 60.5% | 66.5% | 71.5% |
| Organic phase | 37.4% | 32.7% | 29.9% | 27.9% | 25.4% | 16.7% |
| Other phase(s) | 62.6% | 67.3% | 70.1% | 72,2% | 74.6% | 83.3% |
| Number of phases | 3 | 3 | 3 | 3 | 2 | 2 |
| Solid present (salt | Yes | Yes | Yes | Yes | No | No |
| precipitate) | | | | | | |

The organic layer mass was found to decrease with increasing water content in the solution prior to acid addition. At a water content of above 60 wt. % in the feed solution, the ammonium sulfate concentration in the aqueous layer no longer exceeded the solubility limit. The aqueous layer then only contained a single, liquid phase and no solid precipitate. Under these conditions, the loss of acids to the aqueous layer became significant. Thus, control of the water solution in the initial feedstock is shown to be critical to acid recovery.

Table 5 shows the component (e.g. butyric and acetic acid) mass split between the organic and aqueous layers as a function of water content in the solution prior to acid addition. Note that at water contents of 55% or less, the butyric acid retention in the organic layer was greater than 90%. At a water content of 55%, the acetic acid was split almost equally between the aqueous and the organic layer. Even at a water content of 45%, the acetic acid retention was split 66% and 34% between the organic and aqueous layers, respectively. The process therefore favors recovery of butyric acid in the organic layer.

TABLE 5
Mass split

| Water content | | | | | | |
|---|---|---|---|---|---|---|
| in feed | 45.4% | 51.8% | 55.6% | 60.5% | 66.5% | 71.5% |
| Organic phase | | | | | | |
| Butyric acid | 95.4% | 94.6% | 91.3% | 87.0% | 78.2% | 61.0% |
| Acetic acid | 67.2% | 61.1% | 51.2% | 47.0% | 39.9% | 24.3% |
| Lactic acid | 29.5% | 25.4% | 20.3% | 19.4% | 18.6% | 11.5% |
| Water | 16.0% | 13.5% | 13.7% | 13.9% | 14.0% | 9.1% |
| Aqueous phase | | | | | | |
| (liquid) | | | | | | |
| Butyric acid | 4.6% | 5.4% | 8.7% | 13.0% | 21.8% | 39.0% |
| Acetic acid | 32.8% | 38.9% | 48.8% | 53.0% | 60.1% | 74.7% |
| Lactic acid | 70.5% | 75.5% | 79.7% | 80.6% | 81.4% | 88.5% |

Table 6 shows the composition of the organic layer as a function of starting solution water content. It is noted that while the butyric acid mass split shifts to the aqueous layer with increased water content, the fraction of butyric acid in this layer remains relatively constant. With increasing water content, there is some dilution of the acids by the water; however, this dilution is modest in the range of 45 to 55 wt. % water content in the feed, corresponding to a butyric content of 59.7% to 58.7% in the respective range.

TABLE 6
Organic phase composition

| Water content in | | | | | | |
|---|---|---|---|---|---|---|
| feed | 45.4% | 51.8% | 55.6% | 60.5% | 66.5% | 71.5% |
| Butyric acid | 59.7% | 61.0% | 58.7% | 55.9% | 52.4% | 52.6% |
| Acetic acid | 17.2% | 15.7% | 13.0% | 12.3% | 10.9% | 8.9% |
| Lactic acid | 0.6% | 0.5% | 0.5% | 0.4% | 0.5% | 0.4% |
| Water | 21.9% | 22.2% | 27.3% | 30.9% | 35.9% | 37.8% |

### Example 3

Feedstock was subjected to serial dilution and acidulated to a pH of less than 2.0 with 93 wt % concentrated sulfuric acid. One set of samples was maintained at 15° C. while another series of samples was maintained at 65° C.

Table 7 shows the composition of diluted samples used both at 15° C. and 65° C. These compositions were measured prior to acid addition.

TABLE 7
Composition (wt. %)

| | | | | |
|---|---|---|---|---|
| Water | 45% | 50% | 55% | 60% |
| Ammonium | 36.34% | 31.90% | 29.22% | 25.87% |
| butyrate | | | | |
| Butyric acid | 0.29% | 0.43% | 0.57% | 0.62% |
| Ammonium acetate | 16.40% | 14.46% | 13.26% | 11.77% |
| Acetic acid | 0.10% | 0.15% | 0.19% | 0.21% |
| Ammonium lactate | 1.44% | 1.28% | 1.18% | 1.05% |
| Lactic acid | 0.00% | 0.00% | 0.00% | 0.00% |
| Water | 45.44% | 51.78% | 55.59% | 60.48% |

Table 8 shows the organic phase's component mass fraction for the 15° C. samples.

TABLE 8
Component mass split at 15° C.

| | | | | |
|---|---|---|---|---|
| Water content in feed | 45% | 50% | 55% | 60% |
| Butyric acid | 95.4% | 94.6% | 91.3% | 87.0% |
| Acetic acid | 67.2% | 61.1% | 51.2% | 47.0% |
| Lactic acid | 29.5% | 24.5% | 20.3% | 19.4% |
| Water | 16.0% | 13.5% | 13.7% | 13.9% |

Table 9 shows the organic phase's component mass fraction for the 65° C. samples.

TABLE 9
Component mass split at 65° C.

| | | | | |
|---|---|---|---|---|
| Water content in feed | 45% | 50% | 55% | 60% |
| Butyric acid | 95.5% | 95.2% | 91.1% | 83.3% |
| Acetic acid | 69.7% | 61.9% | 54.2% | 41.8% |
| Lactic acid | 32.9% | 26.5% | 23.3% | 19.4% |
| Water | 21.3% | 11.0% | 14.1% | 14.5% |

Butyric acid retention in the organic layer was nearly unchanged over the range from 45 to 60 wt. % water and was not strongly influenced by temperature variation. Acetic acid, on the other hand, shifted to the aqueous phase over this feedstock water range. Further at the higher end of the water range, a higher temperature resulted in greater movement of acetic acid into the aqueous phase.

### Example 4

As shown in Example 2, starting water contents in excess of 60% began to result in significant loss of butyric acid into the aqueous layer.

In the present example, ammonium sulfate salt was added to a 65 wt. % solution to bring the effective water content of the mixture to 50%. The mixture was then acidulated with 93 wt % concentrated sulfuric acid to a pH less than 2.0. One sample was maintained at 13° C., while another sample was maintained at 63° C.

Table 10 shows component recovery in the organic layer for the two temperatures. Butyric acid retention in the organic phase was equivalent to that of a lower water content feedstock. Thus, this experiment showed that salt addition can be employed in an equivalent manner to increasing water evaporation in the feedstock.

It was noted that the experiment also showed almost complete insensitivity to temperature variation. Further, the organic phase had a particularly low water content, a net positive impact of the salt addition. Less water being carried forward into distillation and subsequent refinement lead to higher yield of butyric acid. There exists a water and butyric acid azeotrope which contributes to butyric acid yield loss on subsequent distillation.

TABLE 10
Organic phase recovery

| Temperature at | | |
|---|---|---|
| acidulation | 13° C. | 63° C. |
| Butyric acid | 90.7% | 90.0% |
| Acetic acid | 45.3% | 43.7% |
| Lactic acid | 17.2% | 17.3% |
| Water | 9.3% | 9.5% |

1. A method for producing butyric acid, comprising
(i) providing a fermentation liquor comprising water, butyrate, acetate, at least one counter ion, and at least one fermentation co-product, wherein (a) a butyrate to acetate weight/weight ratio is in the range between 2 and 20, (b) a pH is in the range between 4.5 and 8.0, and (c) a combined concentration of butyrate and acetate is in a range between 20.0 and 75.0 wt. %;
(ii) providing a mineral acid solution comprising said mineral acid and water, wherein (a) the concentration of the mineral acid is at least 70 wt % and (b) said mineral acid is characterized by forming with said counter-ion a first mineral salt with water solubility at 25° C. greater than 25 wt %;
(iii) combining said provided fermentation liquor with said provided mineral acid, whereby an acidulation product is formed; which acidulation product comprises at least two phases, an organic phase comprising butyric acid, acetic acid and water, and an aqueous phase comprising water and the first mineral salt of said acid and the counter ion, wherein a pH of said aqueous phase is in a range between 2.0 and 4.7;
(iv) separating said two phases to form a separated organic phase and a separated aqueous phase; and
(v) separating butyric acid from said separated organic phase to form separated butyric acid.

2. The method of claim 1, wherein the combined amount of water in said provided fermentation liquor and said provided mineral acid is such that the concentration of said first mineral salt in the acidulation product is at least 80% of saturation at 25° C.

3. The method of claim 1, wherein a water content in said provided fermentation liquor is in the range of 40-50%.

4. The method of claim 1, wherein said providing said fermentation liquor comprises fermenting a carbon source with cells of a microorganism capable of producing butyrate and acetate at pH in a range between 4.5 and 7.5 and separating the cells of said microorganism to form said fermentation liquor.

5. The method of claim 4, further comprising removing water from said fermentation liquor.

6. The method of claim 1, wherein said acidulation product further comprises crystals of said first mineral salt and wherein said aqueous phase is saturated with said first mineral salt.

7. The method of claim 1, further comprising adding a second mineral salt to said fermentation liquor such that the mixture of the combined fermentation liquor and second mineral salt has a water content of less than 60 wt. %.

8. The method of claim 7, wherein said second mineral salt and said first mineral salt are a same mineral salt.

9. The method of claim 1, further comprising refining said separated organic phase prior to said separating butyric acid.

10. The method of claim 1, wherein said separating butyric acid comprises distillation, extraction or a combination thereof.

11. The method of claim 1, further comprising removing water from said separated butyric acid.

12. The method of claim 1, further comprising separating acetic acid from said separated organic phase to form separated acetic acid.

13. The method of claim 1, further comprising separating said first mineral salt from said separated aqueous phase.

14. The method of claim 5, further comprising separating said crystals from said acidulation product to form separated crystals of said first mineral salt.

15. The method of claim 1, wherein said counter ion is ammonium, wherein said mineral acid is sulfuric and wherein said first mineral salt is ammonium sulfate.

16. The method of claim 1, further comprising reacting said separated butyric acid or a product thereof with hydrogen to form butanol.

17. A product comprising the separated butyric acid of claim 1.

18. A product comprising the separated acetic acid of claim 12.

19. A product comprising the separated crystals of said first mineral salt of claim 14.

20. An animal feed ingredient comprising the product of claim 19.
